(19) United States
(12) Patent Application Publication
(10) Pub. No.: US 2004/0224024 A1
(43) Pub. Date: Nov. 11, 2004

(54) Controlled drug release formulations containing polyion complexes

(75) Inventors: Noriho Kamiya, Fukuoka (JP); Alexander M. Klibanov, Newton, MA (US)

Correspondence Address: PATREA L. PABST, PABST PATENT GROUP LLP, 400 COLONY SQUARE, SUITE 1200, ATLANTA, GA, 30361, US

(73) Assignee: Massachusetts Institute of Technology

(21) Appl. No.: 10/831,492

(22) Filed: Apr. 23, 2004

Related U.S. Application Data
(60) Provisional application No. 60/465,285, filed on Apr. 23, 2003.

(51) Int. Cl. A61K 38/00; A61K 9/14

(52) U.S. Cl. 424/486; 514/2

(57) ABSTRACT

A composition containing a water-insoluble complex formed of a charged drug and an oppositely charged polyion and the method of making and using the composition are provided. The composition provides sustained release of the charged drug over a period ranging from hours to days. The charged drug can be any natural or synthetic drug. Preferably, the charged drug is a natural or recombinant peptide, polypeptide, or protein. The composition can be formulated into a variety of formulations. Rate of release is controlled by selection of the charge strength and molecular weight of the polyion.

### STATEMENT REGARDING FEDERALLY FUNDED RESEARCH

The Federal Government has certain rights in the invention disclosed herein by virtue of Grant No. GM26698 to Alexander Klibanov from the National Institute of Health.

### FIELD OF THE INVENTION

The present invention is in the field of controlled release of drugs, and in particular, relates to controlled release of a drug such as a peptide, polypeptide, polynucleotide, or protein from water-insoluble polyelectrolyte complexes.

### BACKGROUND OF THE INVENTION

This application claims priority to U.S. Ser. No. 60/465,285 filed Apr. 23, 2003.

Over a hundred pharmaceuticals have been either approved for clinical use or are undergoing clinical trials (Struck MM, “Biopharmaceutical R&D success rates and developments times: a new analysis provides benchmarks for the future” in Bio/Technology 12:674-677 (1994)). The majority of peptides and protein drugs have to be delivered to a patient by injection due to very low oral and transdermal bioavailabilities (Wallace BM, Lasker JS, “Stand and deliver: getting peptide drugs into the body” in Science 260:912-913 (1993)). Due to the short half-life in serum of most peptides, hours or even minutes, they are rapidly cleared from systemic circulation, necessitating frequent injections and, consequently, poor patient compliance (Burke P A, “Controlled release protein therapeutics: effects of process and formulation on stability” In: Wise DL, executive editor; Handbook of Pharmaceutical Controlled Release Technology; M. Dekker, New York, p 661-692 (2000)).

The rapid clearance of protein therapeutics from the blood stream can be decreased by entrapping them within polymeric controlled release matrices (Cleland JL, Langer RS, (Eds.), Formulation and Delivery of Proteins and Peptides, American Chemical society, Washington, D.C (1994)). This methodology, pioneered by Langer and Folkman (Langer R, Folkman J., “Polymers for the sustained release of proteins and other macromolecules” in Nature 263:793-800 (1976)), affords continuous protein release for up to several months (Burke, 2000) but is often plagued by protein aggregation within sold polymeric particles. Alternatively, therapeutic proteins can be covalently derivatized with water-soluble polymers, such as poly(ethylene glycol), to improve their pharmacokinetics and pharmacodynamics (Kung AHC, Baughman RA, Larrick JW, (Eds.), Therapeutic Proteins: Pharmacokinetics and Pharmacodynacis, Freeman, N.Y.(1993)) but this irrevocably alters the protein and creates a new chemical entity, requiring separate approvals by the Food & Drug Administration (“FDA”).

Drug delivery formulations consisting of the peptide or protein in the form of a salt or ionically bound to a polymer to form a water soluble complex have been described. See, for example, U.S. Pat. Nos. 5,889,110 and 6,034,175, which describe the formation of salts of peptides with hydrophobic carboxy-terminated polyesters for sustained release of the peptide drug. The rate of release is controlled by the degradation rate of the hydrophobic polyester. U.S. Pat. No. 5,188,825 describes using weak polycation or weak polyanion exchange resin to bind a water soluble active agent to provide sustained release of the water soluble active agent. This suffers from the disadvantage of very weak binding of drug to the polyion.

Therefore, there is a need for protein delivery formulations that provide controlled release of the protein molecules without aggregation and without covalent interaction between the protein and carrier.

It is an object of the present invention to provide a drug delivery composition that provides a controlled release of the drug, without aggregation or covalent interaction between the drug and carrier.

It is a further object of the present invention to provide a protein composition in which the protein molecules do not aggregate within the carrier.

### SUMMARY OF THE INVENTION

A composition containing a water-insoluble complex formed of a charged drug and a strong polyion and the method of making and using the composition are provided. The composition or water-insoluble complex provides a sustained release of the charged drug over a period of 0-96 hours.

The charged drug can be any natural or synthetic drug. Preferably, the charged drug is a natural or recombinant peptide, polypeptide, or protein, such as a monoclonal antibody, an enzyme, a hormone, or other biologically active and/or therapeutically useful substance.

The polyelectrolyte is a polyion or polycation. The rate of release can be controlled by selection of the charge strength of the polyion. Weak polyanions typically include carboxylic acid groups. Strong polyanions typically include sulfonic acid groups, phosphonic acid groups, phosphonic groups or sulfate groups. The composition can be formulated into a variety of formulations. Representative formulations include, for example, tablets, capsules, microparticulate formulations, hydrogel, hydrogel particles, and emulsions. The microparticulate composition can have particles of sizes ranging from 1 nm to 1 mm. Hydrogel particles have a size ranging from 1 μm to 10 mm. The composition may further include other pharmaceutically acceptable carriers or excipients.

### BRIEF DESCRIPTION OF DRAWINGS

FIG. 1 is the time course (hours) of lysozyme release (percentage) into solution from its water-soluble complexes with weak polyacids-carboxymethylcellulose (CMC) (a); alginate (b); poly-L-aspartate (c); polyacrylate, average molecular weights of 1,200 daltons (d); polyacrylate, average m.w. 5,000 daltons (e); polyacrylate, average m.w. 90,000 daltons (f); and polymethacrylate (g).

FIG. 2A is the time course (hours) of lysozyme release (percentage) into solution from its water-insoluble complexes with polysulfates: heparin [average molecular weights of 3,000 daltons (a), open circles, and 131,000 daltons (b), closed triangles], dextran sulfate [average molecular weights of 8,000 daltons (c), open triangles and 500,000 daltons (d), closed squares], and polyvinylsulfonate (e), closed circles. FIG. 2B is the time course (hours) of lysozyme release (percentage) into solution from its water-insoluble complexes with polyphosphates: double-stranded (a), dark diamonds, and single-stranded (b), open diamond.

FIG. 3 shows the dependence of the rate of release of lysozyme (micrograms protein/ml-min) from spherical calcium alginate hydrogel particles as a function of the cubic root of the number of particles.

### DETAILED DESCRIPTION OF THE INVENTION

There are two principal components of the drug delivery formulation consisting of a water insoluble complex: a charged drug and a polyion (i.e., polyanion or polycation). The water-insoluble complex provides sustained release of the charged drug over a period of from, for example, 0-96 hours, 0-72 hours, 0-48 hours, 0-24 hours, 0-12, or 0-6 hours. The water-insoluble complex also can be formulated into microparticulate sustained release formulations with one or more polymeric excipients to provide sustained release of the charged drug over a period of from, for example, about 0-100 days, 0-60 days, 0-30 days, 0-14 days, 0-7 days, or 0-5 days.

I. Water-Insoluble Complex Composition

A. Drugs to be Delivered

The release rate of the drug is controllable by the ionic strength and molecular weight of the polyelectrolyte rather than by the degradation of the polyelectrolyte. The charged drug can be any biologically active agent that bears a countercharge(s) to the polyelectrolyte. The charged drug molecule can be any biologically active molecule, particularly pharmaceutically active molecule, bearing a charge. The charge can be cationic or anionic. Representative examples of the biologically active agent include protein, peptide, polypeptide, DNA, nucleic acid, nucleoside, polysaccharide, and synthetic or natural organic or inorganic molecules.

In one embodiment, the charged drug is protein, peptide, or polypeptide that bears a positive charge under the physiological or similar condition, for example, a solution at a pH in the range from about pH 4 to about pH 8, preferably from about pH 6 to about pH 8, more preferably from about pH 7 to pH 8. The peptide, polypeptide, or protein can be natural or recombinant. Representative useful proteins include, for example, monoclonal antibodies, insulin, interferons, blood factors, cytokines, therapeutic enzymes, and EPO.

B. Polyions (Polyanions or Polycations)

Any strong biocompatible polyion is useful for forming the water-insoluble complex described herein. The polyion can be synthetic, which include inorganic or organic, and natural polyions. The polyions can have molecular weights in the range, for example, from about 1,000 Daltons to about 1,000,000 Daltons, about 1,000 Daltons to about 500,000 Daltons, about 3,000 Daltons to about 500,000 Daltons, or about 5,000 Daltons to about 100,000 Daltons. A polyion having a particular weight average molecular weight is selected according to the properties of the charged drug molecule and the type of the repeating units in the polyion. Higher-molecular-weight polyions tend to “wrap around” the oppositely charged drug, thereby retarding its releases.

Weak acid groups include, for example, carboxylate groups. Strong acid groups include, for example, sulfonate groups, sulfate groups, nitrate groups, phosphonic and phosphate groups. Representative useful strong polyanions include those shown in the lower half of Table I, below, for example, polyvinylsulfonate, heparin, dextran sulfate, single and double stranded DNA and RNA. Weak polyanions include carboxymethylcellulose, alginate, poly-L-aspartate, polyacrylate, polymethacrylate. Moreover, it is possible to combine a weak polyanion with strong polyanions to make a formulation having the desired release properties.

II. Pharmaceutical Formulations

The water-insoluble complex can be generated by mixing the polyion and the charged drug under agitation or any other methods known in the art of drug formulation.

The water-insoluble complex can be formulated into formulations of the complex per se or in combination with other pharmaceutically acceptable excipients and/or a pharmaceutically acceptable carrier.

The water-insoluble complex can be formulated into tablets, capsules, emulsions, hydrogels, or microparticulate formulations such as microcapsules, microparticles or nanoparticles using known techniques. Methods of preparing these various formulations have been described in, for example, U.S. Pat. Nos. 6,156,339; 5,837,287; 5,827,541; 5,729,958; 5,046,618; 5,343,672; 5,358,118; and 5,188,825.

The microparticulate formulation of the water-insoluble complex includes, for example, a plurality of particles of the water-insoluble complex having a size in the range from, for example, about 1 nm-500 μm, about 100 nm-100 μm, about 1 μm-100 μm, or about 10 μm-100 μm.

The hydrogels can be used as such or as hydrogel particles having a size ranging from, for example, 1 μm to 10 mm, preferably from 10 μm to 1 mm, more preferably from about 100 μm to 500 μm. Hydrogel particles can be prepared by extrusion of the complex into a solution of an appropriate pH, thereby forming particles of various sizes using, for example, a syringe equipped with a needle which can have a variable gauge-number. For example, gel particles smaller than approximately 2.3 mm in diameter can be prepared using a syringe equipped with a 21-, 23-, or 24-gauge needle. Larger hydrogel particles can be prepared using, for example a pipette by changing the inner tip diameter from between, for example, 0.6 mm and 6.0 mm.

Pharmaceutically Acceptable Excipients

Optional pharmaceutically acceptable excipients include, but are not limited to, diluents, binders, lubricants, disintegrants, colorants, stabilizers, surfactants and the like. Diluents, also termed “fillers,” are typically necessary to increase the bulk of a solid dosage form so that a practical size is provided for compression of tablets or formation of beads and granules. Suitable diluents include, for example, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar. Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Lubricants are used to facilitate tablet manufacture; examples of suitable lubricants include, for example, magnesium stearate, calcium stearate, stearic acid, glycerol behenate, and polyethylene glycol, talc, and mineral oil. Disintegrants are used to facilitate dosage form disintegration or “breakup” after administration, and are generally starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp). Stabilizers are used to inhibit or retard drug decomposition reactions which include, by way or example, oxidative reactions. Surfactants may be anionic, cationic, amphoteric or nonionic surface active agents. Suitable anionic surfactants include, but not limited to those containing carboxylate, sulfonate and sulfate ions. Examples for anionic surfactants are sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but not limited quaternary ammonium compounds such as benzalkonium chloride, benzethonium chloride, cetrimonium bromide, stearyl dimethylbenzyl ammonium chloride, polyoxyethylene (15) and coconut amine. Examples for nonionic surfactants are, but not limited to, ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene (8) monolaurate, polysorbates, ii polyoxyethylene (9) octylphenylether, PEG-1000 cetyl ether, polyoxyethylene (3) tridecyl ether, polypropylene glycol (18) butyl ether, Poloxamer 401, stearoyl monoisopropanolamide, and polyoxyethylene (5) hydrogenated tallow amide. Examples for amphoteric surfactants are, but not limited to, sodium N-dodecyl-.beta.-alanine, sodium N-lauryl-.beta.-iminodipropionate, myristoamphoacetate, lauryl betaine and lauryl sulfobetaine. If desired, the formulation may also contain minor amount of nontoxic auxiliary substances such as wetting or emulsifying agents, pH buffering agents, and/or preservatives.

Polymeric excipients for sustained release formulations include natural and synthetic polymers. Synthetic polymers that can be used include bioerodible polymers such as poly(lactide) (PLA), poly(glycolic acid) (PGA), poly(lactide-co-glycolide) (PLGA), and other poly(alpha-hydroxy acids), poly(caprolactone), polycarbonates, polyamides, polyanhydrides, polyamino acids, polyortho esters, polyacetals, polycyanoacrylates and degradable polyurethanes. Examples of natural polymers include proteins such as albumin, collagen, synthetic polyamino acids, and prolamines, and polysaccharides such as alginate, heparin, and other naturally occurring biodegradable polymers of sugar units. Another class of useful excipients are diketopiperazines described in, for example, U.S. Pat. Nos. 5,352,461, 5,503,852, 6,071,497, 5,877,174, 6,153,613, 5,693,338, 5,976,569, 6,331,318 and 6,395,774.

The content of the charged drug in the formulation varies with the molecular weight of the polyion in the water-insoluble complex and the particular type of formulation. Typically, the load of the charged drug can be in the range from, for example, about 0.01 wt % to about 70 wt %, or about 1 wt %, 5 wt %, 10 wt %, 15 wt %, 20 wt %, 25 wt %, 30 wt %, 35 wt %, 40 wt %, 45 wt %, 50 wt %, 55 wt %, 60 wt % or 65 wt % to about 70 wt %. One of ordinary skill in the art of drug formulation would be able to determine a proper load of the charged drug as water-insoluble complex for the treatment of a particular disease.

III. Method of Using the Water-Insoluble Complex

Generally, a composition containing an effective amount of the charged drug in the form of water-insoluble complex is administered to a human or animal in need of treatment or prevention of a disease or condition, based on the drug to be delivered. Depending on the formulation and drug, the complex can be administered parenterally or enterally. For example, the drug formulation may be orally administered as a capsule or tablet. A polymeric implant or microparticles may be injected intravenously, subcutaneously, inhaled through the pulmonary or nasal system, or formulated in an ointment, gel, cream, or other topical or mucosal formulation.

The present invention will be further understood by reference to the following non-limiting examples.

### EXAMPLES

### Example 1

### Release of Lysozyme from Lysozyme-Polyanion Complexes

Materials and Methods

Materials

Hen egg-white lysozyme (58,100 units/mg solid), lyophilized cells of Micrococcus lysodeikticus, carboxymethylcellulose (CMC) (Na salt, low viscosity), dextran and diethylaminoethyl (DEAE)-dextra (MW approx. 500,000 for both), dextran sulfates (Na salt, MW approx. 8,000 and 500,000), heparin (Na salt), low-molecular-weight heparin (Na salt, from porcine intestinal mucosa, MW approx. 3,000), poly-L-aspartic acid (Na salt, MW 15,000-50.000), salmon testes DNA (Na salt) and its single-stranded form (10 mg/ml in water) were all purchased from Sigma Chemical Co. (St. Louis, Mo.). Alginic acid (Na salt), poly(acrylic acid)s [average MW 1,200 (Na salt), 5,000 (partial Na salt) and 90,000], and polyvinylsulfonic acid) (Na salt) were from Aldrich Chemical Co. Poly(methacrylic acid) (average MW 100,000) was from Polysciences (Warrington, Pa.). All chemicals were of analytical grade and used without further purification.

Precipitation of Lysozyme with Polyanions

Precipitation of lysozyme with polyanions was carried out as follows. One ml of a solution of a polyanion in 10 Memorandum phosphate buffer (pH 7.0) was diluted 5 fold with an aqueous solutions of lysozyme in the same buffer. The final concentrations of lysozyme and polyanions were adjusted to 3.0 mg/ml and 5 Memorandum (calculated on the monomeric unit basis), respectively. After a 1-min stirring, a suspension formed was left for 2 h at room temperature to complete the phase separation and then centrifuged at 10,000 rpm and room temperature for 10 min (Rotor SS-34, Sorvall RC-5B Centrifuge, DuPont Instruments). In the case of double-stranded DNA, 1.5 ml of a Ph 7.0 buffered aqueous solution of lysozyme (10 mg/ml) was added to 3.5 ml of a solution of DNA (2.3 mg/ml) in the same aqueous buffer to reduce the viscosity of the solution. Vigorous agitation of the mixture led to the formation of gel-like fibrous precipitates.

The precipitation yields were calculated by comparing the enzymatic activity of lysozyme and the protein concentration in the supernatant with the control sample (3.0 mg/ml lysozyme in the same aqueous buffer) in each experiment. Lysozyme was assayed on the basis of its ability to lyse dried M. lysodeikticus cells (Shugar, 1952: Rozema and Gellman, 1996). Protein concentration was evaluated by absorbance at 282 nm (A₂₈₂). All spectrophotometric measurements were carried out using a Hitachi U-3110 spectrophotometer.

Lysozyme Release from Polyanion Complexes

Polyanion complexes (i.e., water-insoluble precipitates recovered by centrifugation) were resuspended in aqueous pH 7.4 PBS to give a final concentration of 0.03 mg/ml lysozyme and shaken at 37° C. and 150 rpm. Periodically, 100-μl aliquots were withdrawn, centrifuged, and assayed. In the case of 500-kD dextran sulfate-lysozyme complex, 150-μl aliquots were required because of the low amount of lysozyme released. Lysozyme activity in the supernatant was determined as described above and converted to the concentration of lysozyme based on the standard calibration curve separately obtained with the native enzyme. All experiments were done at least in triplicate.

Preparation of Lysozyme-Containing Calcium Alginate Hydrogel Particles

Formation of calcium alginate gel particles was carried out following a procedure of Bucke (1987). A 2% aqueous sodium alginate solution (4.5 ml) and 5 mg/ml aqueous solution of lysozyme (0.5 ml) were prepared in 10 Memorandum Tris-HCl buffer (pH 7.0) and vigorously stirred to yield a homogeneous translucent solution. The hydrogel particles were prepared by extruding it dropwise into 50 ml of a 0.1 M CaCl₂ solution in the same buffer containing 0.5 mg/ml lysozyme under gentle agitation at room temperature, followed by curing in that solution for 2 h. The gel particles smaller than approximately 2.3 Memorandum in diameter were prepared using a syringe equipped with a 21-, 23-, or 24-gauge needle. Larger particles were prepared using a pipette by changing the inner tip diameter from 0.6 to 6.0 mm by cutting the edge. From 5 ml of the alginate solution of lysozyme, batches of 45, 68, 94, 130, 198, 255, 309, 396, and 449 of hydrogel particles were thus prepared. The particle size was measured using a Leica MZ8 stereomicroscope.

Lysozyme Release from Hydrogel Particles

The release of lysozyme from the calcium alginate gel particles was monitored in pH 7.4 Tris-HCl buffered saline (TBS). The particles were recovered by filtration and wiped out with Kimwipes® prior to release experiments to remove lysozyme solution microdoplets from the surface. The particles were placed in TBS (50 ml), agitated at 37° C. and 150 rpm, and 0.5-ml supernatant aliquots were withdrawn for up to 5 min at 1-min intervals. The initial release rates were measured by monitoring the increase in the activity of lysozyme in the medium up to 15% of the total amount of lysozyme entrapped. The release rates obtained were plotted against the cubic root of the number of particles and fitted to the y=a x equation by the least-square method using the Sigma Plot 5.0 scientific software package (Statistical Product & Service Solutions).

TABLE I
Precipitation of lysozyme from aqueous solution due to water-insoluble
complex formation with polyanions^{a}

| | Average | Number of | | |
|---|---|---|---|---|
| | molecular | monomeric units | % of precipitation | |
| Polyanion | weight, daltons | per chain | A | B |
| Weak acids | | | | |
| carboxymethylcellulose | ˜90,000 | ˜450° | 59 ± 5.1 | f |
| alginate | 12,000-80,000 | 69-460 | 83 ± 3.0 | f |
| Poly-L-aspartate | 15,000-50,000 | 130-440 | 97 ± 1.5 | 96 ± 0.1 |
| polyacrylate | 1,200 | 17 | 97 ± 0.24 | 96 ± 1.2 |
| ″ | 5,000 | 70 | 99 ± 0.22 | 98 ± 0.43 |
| ″ | 90,000 | 1300 | 94 ± 2.0 | 92 ± 2.8 |
| polymethacrylate | 100,000 | 1200 | 99 ± 0.78 | 99 ± 0.95 |
| Strong Acids | | | | |
| polyvinylsulfonate | 4,000-6,000 | 37-56 | 99 ± 0.04 | 98 ± 0.86 |
| Heparin | 3,000 | 11^{e} | 98 ± 1.8 | 96 ± 1.8 |
| ″ | 13,000^{b} | 46^{e} | 97 ± 0.16 | 97 ± 0.27 |
| dextran sulfate | 8,000 | 23 | 99 ± 0.12 | 98 ± 0.35 |
| ″ | 500,000 | 1400 | 99 ± 0.13 | 86 ± 1.6 |
| single-stranded DNA | 190,000-270,000 | 590-830 | 64 ± 5.1 | f |
| double-stranded DNA | ˜1,300,000° | ˜2000 | 95 ± 5.8 | g |

A: based on activity.
B: based on A₂₈₂.
^{a}See text for experimental conditions.
^{b}Average molecular weight of commercial heparin (Hileman et al., 1998).
^{c}Average molecular weight for both chains combined
^{d}Assuming that the molecular weight of the monomer unit is 200.
^{e}Assuming that the average molecular weight of the monomer unit is 281 (Heuck et al., 1985).
f Upon the addition of polyanions, the solution turned translucent.
g The accurate quantification was difficult due to the absorbance of residual DNA at 282 nm.

Results and Discussion

Like other proteins, lysozyme can form water-insoluble complexes with polyions of the opposite charge (Dumitriu S, Chornet E., “Inclusion and release from alginate matrices” in Adv Drug Delivery Rev 31:267-285 (1998)). Lysozyme has an isoelectric point of approximately 11 and hence a net positive charge at neutral pH and thus is capable of forming complexes with a variety of both natural and synthetic polyanions. Lysozyme can be precipitated from aqueous solution due to the formation of a water-insoluble polysalt complex and then kinetically examined the release from the latter of the native protein under physiological conditions.

In a typical experiment, to 3 mg/ml lysozyme dissolved in a pH 7.0 buffered aqueous solution at 5 mM polyanion (calculated on the monomeric unit basis) was added, and the mixture was incubated for 2 h at room temperature. In the case of most polyanions used, a white precipitate formed (Table I), which was recovered by centrifugation. The remaining solution was assayed both for the enzymatic activity of lysozyme and for protein content, and the values obtained were compared with those prior to the addition of polyanion. Note that the precipitate formed was due to a polysalt complex formation, as opposed to molecular crowding, because no precipitation was observed with a neutral polymer (500-kD dextran) or a positively charged one (500-kD DEAE-dextran) under otherwise identical conditions.

Inspection of Table I shows that at the experimental conditions used all polyanions precipitated more than one half of the enzyme present, and in 11 out of 14 instances the protein removal efficiency was in the 90+ % range. Moreover, consistent results almost invariably were obtained whether the precipitation of lysozyme was judged on the basis of the decrease in enzymatic activity or in protein concentration, thus validating the measurements. Additionally, all the relatively inefficient lysozyme-precipitating polyanions possess a lower linear charge density (Manning GS, “Limiting laws for equilibrium and transport properties of polyanion solutions” in Sélégny E, editor, Polyelectrolytes, D Reidel Publishing Company, Dordrecht, Holland, p 9-37 (1974))—e.g., compare carboxymethylcellulose (CMC) and alginate with polyaspartate and heparin, or single-stranded with double-stranded DNA.

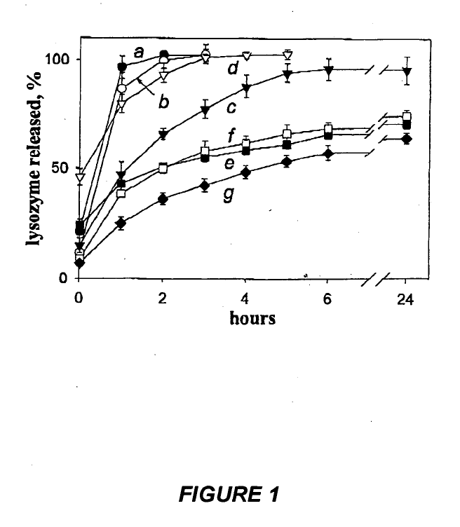

The precipitated polyanion complexes of lysozyme were resuspended (at 0.3 mg/ml) in aqueous pH 7.4 PBS, and the time course of the appearance of the soluble enzymatic activity from them was measured at 37° C. FIG. 1 depicts the release of the enzyme from its complexes with weak polyacids (the upper half of Table I). One can see that the rates of release vary greatly—from the enzyme completely liberated in under 2 h in the case of CMC to less than only two third of the enzyme liberated even after 24 h in the case of polymethacrylate (curves a and g, respectively).

Analysis of the data in FIG. 1 reveals that the rate of release drops as the linear charge density of the polyanion partner is raised. For example, the fastest release was observed with CMC and alginate (curves a and b), the slowest with polymethacrylate and polyacrylate (curves g, e, and f), and an intermediate one with polyaspartate (curve c). In addition, it appears that the increase of the polyion's hydrophobicity favors a slower release—compare polymethacrylate with polyacrylate of a similar molecular weight (curves g and f respectively). These conclusions are consistent with the view that the stronger the lysozyme-polyanion complex (due to enhanced either electrostatic or hydrophobic interactions), the slower the rate of enzyme release.

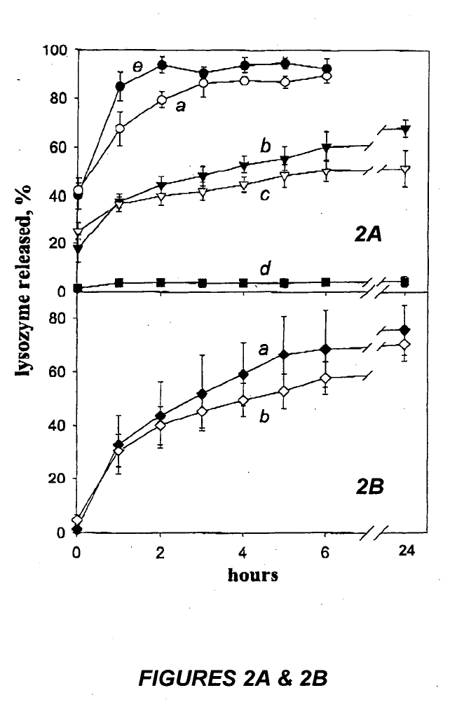

To gain further insights into the factors affecting the release rates, similar studies were conducted with strong polyacids—sulfates (FIG. 2A) and phosphates (FIG. 2B), as opposed to carboxylates (FIG. 1). The data in FIG. 2A point to the effect of molecular weight of the polyanion on the release rate. One can see that lysozyme is released much faster from its complex with 3-kD heparin than with the 13-kD counterpart (curves a and b, respectively). The results are even more dramatic with dextran sulfate: while nearly half of lysozyme is liberated from its complex with the 8 kD polyanion after 24 h, only a few percent is released in the case of 500-kD dextran sulfate (curve c and d, respectively).

These results indicate that if a polyanion is sufficiently long to wrap around the lysozyme molecule, then the resultant complex will be stronger, and hence will afford slower release of lysozyme. Increasing the size of the polyanion, for example, to 90 kD does not further prolong release (curves d, e, and f, respectively). Apparently even the 5-kD polyanion is sufficiently long to wrap around lysozyme, and thus greater still molecular weight has little extra effect. The seemingly contradictory conclusions that while 5-kD polyacrylate is 0.5 long enough to coat the lysozyme molecule 8-kD dextran sulfate is not can be readily rationalized if their numbers of monomeric units (directly related to polymer length), rather than simply molecular weights, are compared —approximately 70 and 23, respectively (3^{rd} column in Table I).

### Example 2

### Effect of Polyanion Complex Particle Size on Release Rate.

Yet another parameter that should affect the rate of lysozyme release is the polyanion complex particle size. One would expect that the rate of protein diffusion out of the insoluble complex should be proportional to the particle's external surface area. If the particles are identical, spherical, and their total volume is fixed, with the only variable being their number N (the larger N, the smaller the particles), then a simple derivation yields the following relationship between the rate of release v and N:

ν=constN^{1/3} (1)

Materials and Methods

In order to verify equation (1) experimentally, lysozyme was entrapped in spherical hydrogel (Chen J, Jo S, Park K., “Polysaccharide hydrogels for protein drug delivery” in Carbohydr Polym 28:69-76 (1995); Hoffman AS, “Hydrogels for biomedical applications” in Adv Drug Delivery Rev 43:3-12 (2002)) particles of calcium alginate (Bucke C., “Cell immobilization in calcium alginate” in Methods Enzymol 135:175-189 (1987); Gombotz and Wee, “Protein release from alginate matrices” in Adv. Drug Delivery Rev. 31:267-285 (1998)). Lysozyme was dissolved in given volumes of a buffered (pH 7.0) aqueous solution of sodium alginate (Table I). Then each volume was separately extruded into a buffered solution of CaCl₂ using needles or pipette tips of different internal diameters (thus resulting in spherical particles of different sizes). In each instance, the hydrogel particles were harvested, counted (giving N), placed in a buffered pH 7.4 aqueous solution, and the rate of lysozyme release was monitored as a function of time at 37° C.

RESULTS

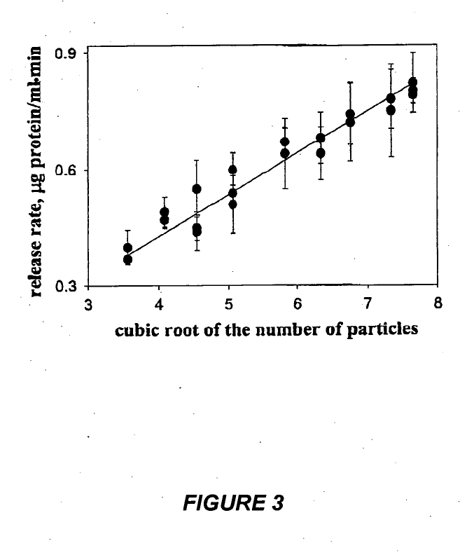

FIG. 3 depicts the combined results of three independent experiments in which the rate of lysozyme release from lysozyme-alginate complex was measured at different N values and plotted in the ν÷N^{1/3} coordinates as prescribed by equation (1). The lysozyme-alginate complex was made out of 5 mL of a lysozyme-containing buffered aqueous solution of sodium alginate. The abscissa is the cubic root of the number of particles to adhere to equation (1). Note that the number of particles made out of given volume was used as a variable, as opposed to the average particle size, because it is more accurate to count particles than to measure their diameter. For reference, the smallest particles (the extreme right points in the figure) were approximately 2.0 mm, and the largest ones (the extreme left points in the figure), were approximately 4.5 mm in diameter. The rate of release was measured as outlined in the legend to FIG. 1 except that the pH 7.4 physiological saline solution was buffered with Tris-HCl rather than phosphate (to avoid calcium phosphate precipitation).

The straight line, drawn by the least-square method, goes through the origin as required by equation (1). It is seen that a good agreement (correlation coefficient of 0.97) is observed between experimental data and the theory expressed by equation (1). This provides yet another way of predictably controlling the rate of protein release.

Publications cited herein and the material for which they are cited are specifically incorporated by reference. Modifications and variations of the present invention will be obvious to those skilled in the art from the foregoing detailed description and are intended to be encompassed by the following claims.

We claim:

1. A formulation for controlled release of a drug comprising a water-insoluble complex formed of a charged drug and a polyion.

2. The formulation of claim 1 wherein the polyion (polyelectrolyte) is a polyanion selected from the group consisting of a polycarboxylate, polysulfate, polyphosphate, and a polysulfonate.

3. The formulation of claim 2 wherein the polyanion is selected from the group consisting of polyvinylsulfonate, heparin, dextran sulfate, DNA, carboxymethyl cellulose, poly(acrylic acid), and carboxymethyl dextran.

4. The formulation of claim 1 wherein the charged drug is a natural or recombinant peptide, polypeptide or protein.

5. The formulation of claim 1 wherein the charged drug is a natural or recombinant polypeptide or protein.

6. The formulation of claim 1 wherein the polyion molecular weight is selected to provide optimal length of release of drug.

7. The formulation of claim 1 further comprising pharmaceutically acceptable excipients.

8. The formulation of claim 7 wherein the water insoluble complex is in a form selected from the group consisting of tablets, capsules, ointments, gels, creams, polymeric implants, particles, powders, and dispersions or suspensions.

9. The formulation of claim 8 wherein the form is hydrogel particles having a size ranging from 1 μm to 10 mm.

10. The formulation of claim 8 comprising a plurality of microparticles having sizes within the range of from 1 nm to 1 mm.

11. The formulation of claim 7 wherein the excipients are selected from the group consisting of sugars, hydrophilic polymers, surfactants, dispersants, pore-forming agents, wetting agents, dispersants, and binders.

12. The formulation of claim 1 wherein the formulation provides a sustained release of the drug over a period of 0-96 hours.

13. The formulation of claim 1 wherein the formulation provides a sustained release of the charged drug over a period of greater than 96 hours to 100 days.

14. A method of making a formulation for controlled release of a drug comprising complexing a charged drug and an oppositely charged polyion to form a water insoluble complex.

15. The method of claim 14 wherein the polyion is selected from the group of polyanions consisting of a polycarboxylate, polysulfate, polyphosphate, and a polysulfonate.

16. The method of claim 14 wherein the polyion is selected from the group consisting of polyvinylsulfonate, heparin, dextran sulfate, and DNA, carboxymethyl cellulose, poly(acrylic acid), and carboxymethyl dextran.

17. The method of claim 14 wherein the charged drug is a natural or recombinant peptide, polypeptide or protein.

18. The method of claim 14 further comprising adding excipients to the water insoluble complex to make a formulation in a form selected from the group consisting of tablets, capsules, ointments, gels, creams, polymeric implants, particles, powders, and dispersions or suspensions.

19. The method of claim 14 wherein the molecular weight of the polyion is selected to optimize the duration of release of the drug.

20. The method of claim 14 comprising adding hydrophobic materials to the formulation to increase the duration of release.

21. The method of claim 14 comprising forming the insoluble complex into particles of a size increasing the duration of release.
